(19) United States
(12) Patent Application Publication
(10) Pub. No.: US 2012/0252087 A1
(43) Pub. Date: Oct. 4, 2012

(54) Bipolar Flyback Power Supply

(75) Inventors: Robert E. Hebner, Austin, TX (US); Mark M. Flynn, Austin, TX (US); Michael D. Werst, Manor, TX (US)

(73) Assignee: Board of Regents, The University of Texas System, Austin, TX (US)

(21) Appl. No.: 13/439,340

(22) Filed: Apr. 4, 2012

Related U.S. Application Data
(60) Provisional application No. 61/471,612, filed on Apr. 4, 2011.

(51) Int. Cl. C12M 1/42; C12N 13/00; H03K 3/00

(52) U.S. Cl. 435/173.7; 307/106; 435/294.1

(57) ABSTRACT

A device, system and method for treating biological cells includes a voltage source, a half-controlled bridge connected to the voltage source, and a load connected across the half-controlled bridge. The half-controlled bridge includes a first switch, a second switch, a first diode and a second diode. The load includes an inductor connected in parallel with a cell or chamber. A controller is connected to the first and second switches and operates the first switch and the second switch to selectively generate one or more bipolar pulses, wherein each bipolar pulse comprises a positive polarity voltage pulse and a negative polarity voltage pulse with a negligible delay between the positive polarity voltage pulse and the negative polarity voltage pulse.

### CROSS-REFERENCE TO RELATED APPLICATIONS

This patent application claims priority to U.S. Provisional Patent Application No. 61/471,612 filed on Apr. 4, 2011 and entitled “Bipolar Flyback Power Supply,” which is hereby incorporated by reference in its entirety.

### TECHNICAL FIELD OF THE INVENTION

The present invention relates in general to the field of electrical lysing of biological cells, and more particularly, to the design of an electrical power supply for producing positive and negative polarity voltage pulses with negligible delay in between when transitioning from one polarity to the other.

### STATEMENT OF FEDERALLY FUNDED RESEARCH

None.

### REFERENCE OF A SEQUENCE LISTING

None.

### BACKGROUND OF THE INVENTION

Without limiting the scope of the invention, its background is described in connection with electrical treatment of algal and other biological cells with the purpose of lysing said cells.

U.S. Patent Application Publication No. 2009/0087900 (Davey and Hebner, 2009) discloses two apparatuses capable of performing electroporation. The first apparatus uses a Marx generator with a substantial change from its original waveform. The second apparatus is a cable pulse device.

U.S. Pat. No. 6,043,066 issued to Mangano and Eppich (2000) describes methods and devices which enable discrete objects having a conducting inner core, surrounded by a dielectric membrane to be selectively inactivated by electric fields via irreversible breakdown of their dielectric membrane. The '066 Patent has applications in the selection, purification, and/or purging of desired or undesired biological cells from cell suspensions. As described therein, electric fields can be utilized to selectively inactivate and render non-viable particular subpopulations of cells in a suspension, while not adversely affecting other desired subpopulations. The cells can be selected on the basis of intrinsic or induced differences in a characteristic electroporation threshold; which can depend, for example, on a difference in cell size and/or critical dielectric membrane breakdown voltage. Effective cell separation can be performed without the need to employ undesirable exogenous agents, such as toxins or antibodies. The relatively rapid cell separation alos involves a relatively low degree of trauma or modification to the selected, desired cells.

The prior art power supplies suffer from pulse amplitude degradation as the generated stream of pulses continues in time and thus are not typically used for more than one pulse (bipolar or unipolar) without subsequently waiting for a significant recharging delay time. As a result, these power supplies are not capable of generating a series of pulses having a negligible delay between each pulse. There is, therefore, a need for a power supply that can generate a series of pulses with a negligible delay between each pulse to provide more efficient lysing of biological cells.

### SUMMARY OF THE INVENTION

The present invention describes an electrical power supply for producing positive and negative polarity voltage pulses with a negligible delay in between when transitioning from one polarity to the other for the purpose of electrically lysing (tearing open) algal and other biological cells. The lysed algal cells release oils and lipids that can be converted to biodiesel, alternative transportation fuels, and other commercially valuable products.

More specifically, the present invention provides a bipolar pulse generator that includes a voltage source, a half-controlled bridge connected to the voltage source, and a load connected across the half-controlled bridge. The half-controlled bridge includes a first switch, a second switch, a first diode and a second diode. The load includes an inductor connected in parallel with a cell or chamber. A controller is connected to the first switch and the second switch. The controller operates the first switch and the second switch to selectively generate one or more bipolar pulses, wherein each bipolar pulse comprises a positive polarity voltage pulse and a negative polarity voltage pulse with a negligible delay between the positive polarity voltage pulse and the negative polarity voltage pulse. The negligible delay can be one microsecond or less, or no delay at all.

In some embodiments, the bipolar pulse generator may also include an energy recovery circuit connected in series with the cell or chamber such that the cell or chamber and the energy recovery circuit are connected in parallel with the inductor. For example, the energy recovery circuit may include a third diode connected in parallel with a third switch, such that the controller operates the third switch to recover an energy stored in the inductor.

In addition, the present invention provides a system for treating biological cells that includes a cultivation tank, a cell or chamber connected to the cultivation tank, a bipolar pulse generator for delivering one or more bipolar pulses to the cell or chamber, and a separation vessel connected to the cell or chamber. The cultivation tank is used to grow the one or more flocculated or unflocculated biological cells in a presence of a medium comprising fresh water, salt water, brackish water, growth medium or a combination thereof and one or more growth factors comprising nutrients, minerals, CO₂, air, light or a combination thereof. The cell or chamber is used for lysing the biological cells to release neutral lipids, proteins, triglycerides, sugars or combinations thereof using one or more bipolar pulses. The bipolar pulse generator includes a voltage source, a half-controlled bridge connected to the voltage source, and a load connected across the half-controlled bridge. The half-controlled bridge includes a first switch, a second switch, a first diode and a second diode. The load includes an inductor connected in parallel with the cell or chamber. A controller is connected to the first switch and the second switch. The controller operates the first switch and the second switch to selectively generate one or more bipolar pulses, wherein each bipolar pulse comprises a positive polarity voltage pulse and a negative polarity voltage pulse with a negligible delay between the positive polarity voltage pulse and the negative polarity voltage pulse. The negligible delay can be one microsecond or less, or no delay at all. The separation vessel is used to separate the released neutral lipids, proteins, triglycerides, sugars or combinations thereof from other released cellular components.

Moreover, the present invention provides a method for treating biological cells by providing one or more flocculated or unflocculated biological cells in a cell or chamber are provided and applying one or more bipolar pulses to the cell or chamber such that the one or more flocculated or unflocculated biological cells are lysed and release neutral lipids, proteins, triglycerides, sugars or combinations thereof. The one or more bipolar pulses are generated by a bipolar pulse generator, which includes a voltage source, a half-controlled bridge connected to the voltage source, and a load connected across the half-controlled bridge. The half-controlled bridge includes a first switch, a second switch, a first diode and a second diode. The load includes an inductor connected in parallel with the cell or chamber. A controller is connected to the first switch and the second switch. The controller operates the first switch and the second switch to selectively generate one or more bipolar pulses, wherein each bipolar pulse comprises a positive polarity voltage pulse and a negative polarity voltage pulse with a negligible delay between the positive polarity voltage pulse and the negative polarity voltage pulse. The negligible delay can be one microsecond or less, or no delay at all. The released neutral lipids, proteins, triglycerides, sugars or combinations thereof are separated from other released cellular components.

### BRIEF DESCRIPTION OF THE DRAWINGS

For a more complete understanding of the features and advantages of the present invention, reference is now made to the detailed description of the invention along with the accompanying figures and in which:

FIGS. 1A-1B illustrate rectangular pulses used in lysing in accordance with the prior art;

FIG. 2 illustrates a bipolar rectangular pulse with a negligible delay in accordance with one embodiment of the present invention;

FIG. 3A is a schematic diagram of a bipolar pulse generator in accordance with one embodiment of the present invention;

FIG. 3B is a schematic diagram of a bipolar pulse generator in accordance with another embodiment of the present invention;

FIG. 3C is a schematic diagram of a bipolar pulse generator in accordance with yet another embodiment of the present invention;

FIGS. 4A and 4B show voltage and current waveforms for the bipolar pulse generator with a third switch held closed and the third switch at 16 μs respectively in accordance with two embodiment of the present invention;

FIG. 5 shows the energy usage, negative pulse time, efficiency and peak Q₁ current versus Lₘ in accordance with one embodiment of the present invention;

FIG. 6 is a block diagram of a system for treating biological cells in accordance with another embodiment of the present invention; and

FIG. 7 is a flow chart of a method for treating biological cells in accordance with another embodiment of the present invention.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not limit the scope of the invention.

The lysing of biological cells is significantly more effective if the applied voltage pulse can transition from a positive to a negative polarity with negligible delay in between. Specifically, a negligible delay is a delay that is much shorter than the effective time contraints of both the material contained within and that outside of the cell membrane. Rapid voltage reversal prevents rearrangement of induced surface charges resulting in a short state of tension or transient mechanical force in the algal and/or other biological cells and large force reversals. The combination leads to lysing of the algal and/or other biological cells. The electromechanical lysing of algal cells by voltage reversals have been previously described by Hebner et al. in U.S. Patent Application Publication No. 2012/0021481, which is incorporated herein by reference in its entirety. Briefly, a rapid voltage reversal is described for a cell lysis process (e.g., an algal cell) is described therein, specifically, FIGS. 10 and 11 of U.S. Patent Application Publication No. 2012/0021481 (and the detailed description thereof) demonstrate the large forces that lyse the cells. Theoretically, the approach is expected to have little influence on electroporation as electroporation time constants are short compared to the reversal time. Existing technology power supplies that require a delay time between pulses cannot be used to provide bipolar pulses with a negligible delay.

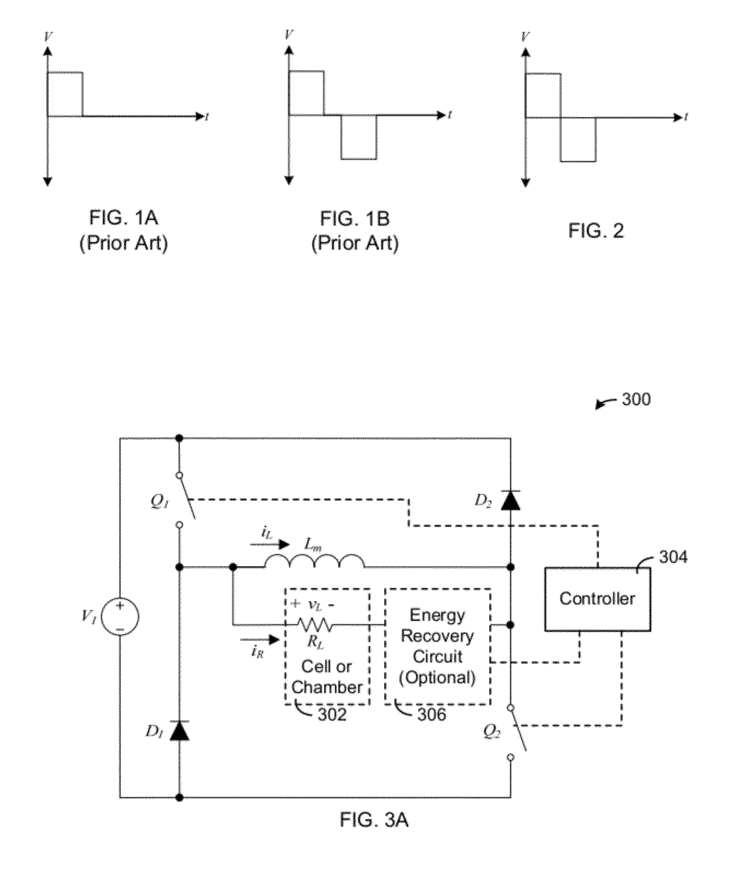

The power supply in accordance with the present invention as described below, also referred to as a bipolar pulse generator or flyback conveter, addresses this issue by describing an apparatus for achieving a rapid voltage reversal with a negiligble delay in between for the purpose of electrically lysing (tearing open) biological cells. In some embodiments, the neglibile delay can mean delay of one microsecond or less, or no delay at all. A system and method using the electrical power supply in accordance with the present invention are also disclosed. The power supply of the present invention is reconfigurable via a user programmable software interface that configures a switch controller in real time to produce a variety of pulse shapes such as (i) bipolar pulses with a negligible delay between the positive and negative pulses (FIG. 2); (ii) bipolar pulses with a specified delay between positive and negative pulses (FIG. 1B—prior art); and (iii) unipolar pulses (positive or negative depending on how the load leads are connected) (FIG. 1A—prior art).

The operation of the three-switch bipolar flyback converter described herein is designed to produce bipolar rectangular voltage pulses across a test cell for the purpose of lysing algal cells contained within to extract the bio-oil [1]. Unlike traditional bipolar pulsed power supplies [2-4], the flyback converter has been designed to provide a rectangular output voltage that is capable of swinging directly from a positive polarity to a negative polarity with equal amplitudes, with a negligible delay in between, using only one voltage source, and without adjusting or reconnecting any of the converter's components. The negligible delay can be one microsecond or less, or no delay at all. Studies with algal cells suggest that the somewhat modest improvement gained when lysing algal cells with a bipolar voltage pulse as compared to a unipolar pulse is greatly enhanced if the dead time between pulses is removed. Thus, the described flyback converter of the present invention is designed to lyse algal cells more effectively than traditional methods which produce unipolar pulses or bipolar pulses with dead time.

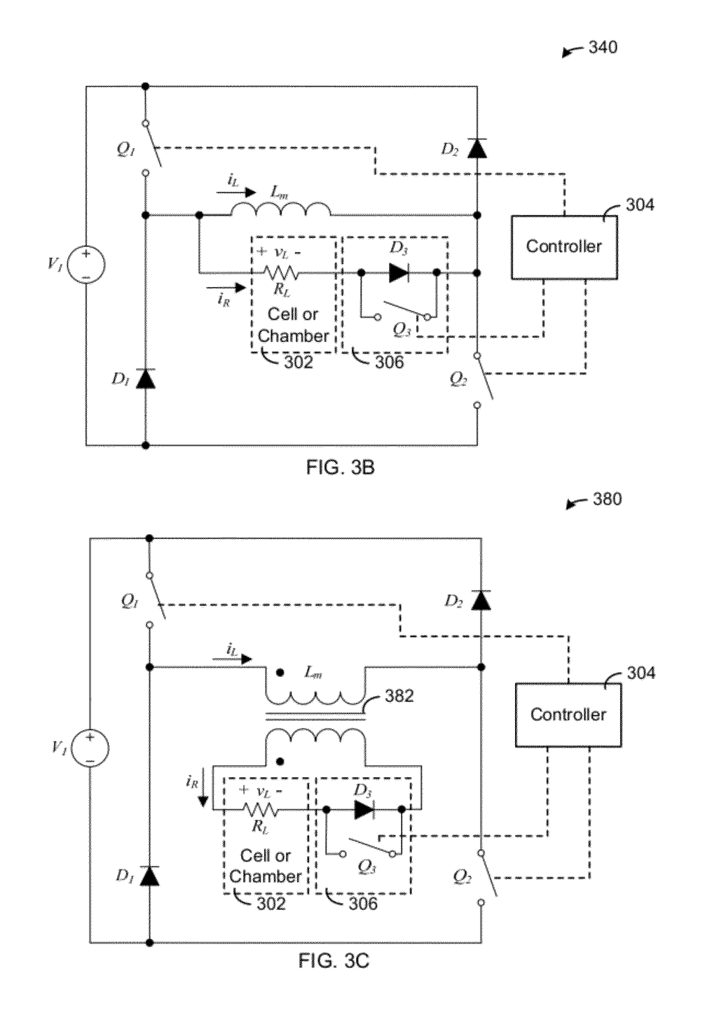

Now referring to FIG. 3A, a schematic diagram of a bipolar pulse generator 300 in accordance with one embodiment of the present invention is shown. The bipolar pulse generator or flyback converter 300 includes a voltage source V₁, a half-controlled bridge connected to the voltage source V₁, and a load connected across the half-controlled bridge. The half-controlled bridge includes a first switch Q₁, a second switch Q₂, a first diode D₁ and a second diode D₂. The load includes an inductor Lₘ connected in parallel with a cell or chamber 302, which can be any type of container or vessel suitable for the purposes described herein. The first switch Q₁ and the second switch Q₂ can be transistors, thyristors or other suitable components. A controller 304 is connected to the first switch Q₁ and the second switch Q₂. The controller 304 operates the first switch Q₁ and the second switch Q₂ to selectively generate one or more bipolar pulses, wherein each bipolar pulse comprises a positive polarity voltage pulse and a negative polarity voltage pulse with a negligible delay between the positive polarity voltage pulse and the negative polarity voltage pulse. The negligible delay can be one microsecond or less, or no delay at all. Moreover, the one or more bipolar pulses can be a continueous stream of bipolar pulses with substantially no voltage degradation of the positive polarity voltage pulse and the negative polarity voltage pulse. As described below, the positive polarity voltage pulse and the negative polarity voltage pulse are preferably approximately rectangular in shape. Note that it is assumed that all circuit components are ideal for the purposes of this description.

The bipolar pulse generator 300 may also include an energy recovery circuit 306 connected in series with the cell or chamber 302 such that the cell or chamber 302 and the energy recovery circuit 306 are connected in parallel with the inductor Lₘ. As will be explained in more detail below, the controller 304 will typically be connected to and control the operation of the energy recovery circuit 306 to recovery energy stored in the inductor Lₘ. Although the energy recovery circuit 306 is not required, it greatly improves the electrical performance and efficiency of the bipolar pulse generator 300. For example, the controller 304 can operate the energy recovery circuit 306 such that approximately 50% of the energy stored in the inductor Lₘ is transferred to the cell or chamber 302 and approximately 50% of the energy stored in the inductor Lₘ is returned to the voltage source V₁.

The bipolar pulse generator 300 can use a single voltage source V₁. In addition, the controller 304 can be programmed using a graphical user interface to operate the first switch Q₁ and the second switch Q₂ selectively generate one or more unipolar pulses, or adjust the opening and closing of the first switch Q₁ and the second switch Q₂ to change a duration of the positive polarity voltage pulse and the negative polarity voltage pulse. Note that the half-controlled bridge can be replaced with a full H-bridge by replacing the first diode D₁ with a fourth switch Q₄ and the second diode D₂ with a fifth switch Q₅ if the components can achieve the negligible delay. Although current switching technology may not be able to achieve the negibile delay using a full H-bridge design, future improvements to switching technology may make this a viable embodiment and is, therefore, within the scope of the present invention.

Referring now to FIG. 3B, a schematic diagram of a bipolar pulse generator 340 in accordance with one embodiment of the present invention is shown. The bipolar pulse generator or flyback converter 340 includes a voltage source V₁, a half-controlled bridge connected to the voltage source V₁, and a load connected across the half-controlled bridge. The half-controlled bridge includes a first switch Q₁, a second switch Q₂, a first diode D₁ and a second diode D₂. The load includes an inductor Lₘ connected in parallel with: (a) a cell or chamber 302 connected in series with (b) an energy recovery circuit 306. The cell or chamber 302 can be any type of container or vessel suitable for the purposes described herein. The energy recovery circuit 306 includes a third diode D₃ connected in parallel with a third switch Q₃. Other circuit configurations for the energy recovery circuit 306 can be used. The first switch Q₁, the second switch Q₂ and the third switch Q₃ can be transistors, thyristors or other suitable components. A controller 304 is connected to the first switch Q₁, the second switch Q₂ and the third switch Q₃. The controller 304 operates: (1) the first switch Q₁ and the second switch Q₂ to selectively generate one or more bipolar pulses, wherein each bipolar pulse comprises a positive polarity voltage pulse and a negative polarity voltage pulse with a negligible delay between the positive polarity voltage pulse and the negative polarity voltage pulse, and (2) the third switch Q₃ to recover an energy stored in the inductor Lₘ. The negligible delay can be one microsecond or less, or no delay at all. Moreover, the one or more bipolar pulses can be a continueous stream of bipolar pulses with substantially no voltage degradation of the positive polarity voltage pulse and the negative polarity voltage pulse. As described below, the positive polarity voltage pulse and the negative polarity voltage pulse are preferably approximately rectangular in shape. Note that it is assumed that all circuit components are ideal for the purposes of this description.

The bipolar pulse generator 340 can use a single voltage source V₁. In addition, the controller 304 can be programmed using a graphical user interface to operate the first switch Q₁ and the second switch Q₂ selectively generate one or more unipolar pulses, or adjust the opening and closing of the first switch Q₁, the second switch Q₂ and the third switch Q₃ to change a duration of the positive polarity voltage pulse and the negative polarity voltage pulse. When the controller 304 opens the third switch Q₃ at an end of a source dominated discharge, approximately 50% of the energy stored in the inductor Lm is transferred to the cell or chamber 302 and approximately 50% of the energy stored in the inductor Lm is returned to the voltage source V₁. Note that the half-controlled bridge can be replaced with a full H-bridge by replacing the first diode D₁ with a fourth switch Q₄ and the second diode D₂ with a fifth switch Q₅ if the components can achieve the negligible delay. Although current switching technology may not be able to achieve the negibile delay using a full H-bridge design, future improvements to switching technology may make this a viable embodiment and is, therefore, within the scope of the present invention.

The cell or chamber 302 may contain an insulation, a biological sample, a medical sample, an environmental sample, an agricultural sample or a combination thereof. Likewise, the cell or chamber 302 may contains one or more biological cells such that bipolar pulses lyse the one or more biological cells such that one or more products, such as neutral lipids, proteins, triglycerides, sugars, and combinations and modifications thereof, are released. The neutral lipids, triglycerides or both can then be converted to yield a fatty acid methyl ester (FAME), a biodiesel or a biofuel. The one or more biological cells can be selected from a domain comprising Prokaryota and/or Eukaryota. The one or more biological cells can be selected from a division comprising Cyanophyta, Archaeplastida/Plantae sensu lato (includes the Phylum Viridiplantae, plants, which includes Chlorophyta, Rhodophyta, and Glaucophyta); Cabozoa (includes the Kingdom Excavata and Supergroup Rhizaria that represents the Euglenophyta and Chlorarachniophyta); Chromaveolata (includes the Supergroup Chromista and 20 Superphylum Aveolata that represent the Heterokontophyta, Haptophyta, Cryptophyta, and Dinophyta), as well as the Kingdom Fungi (all yeasts and fungal-related organisms). Moreover, the one or more biological cells can be algal cells, bacterial cells, viral cells or combinations thereof. Various algal cells are listed below.

Now referring to FIG. 3C, a schematic diagram of a bipolar pulse generator 380 in accordance with another embodiment of the present invention is shown. The bipolar pulse generator or flyback converter 380 includes a voltage source V₁, a half-controlled bridge connected to the voltage source V₁, and a load connected across the half-controlled bridge. The half-controlled bridge includes a first switch Q₁, a second switch Q₂, a first diode D₁ and a second diode D₂. The load includes a transformer 382 (Lₘ) having a primary winding connected across the half-controlled bridge and a secondary winding connected in parallel with: (a) a cell or chamber 302 connected in series with (b) an energy recovery circuit 306. The cell or chamber 302 can be any type of container or vessel suitable for the purposes described herein. The energy recovery circuit 306 includes a third diode D₃ connected in parallel with a third switch Q₃. Other circuit configurations for the energy recovery circuit 306 can be used. The first switch Q₁, the second switch Q₂ and the third switch Q₃ can be transistors, thyristors or other suitable components. A controller 304 is connected to the first switch Q₁, the second switch Q₂ and the third switch Q₃. The controller 304 operates: (1) the first switch Q₁ and the second switch Q₂ to selectively generate one or more bipolar pulses, wherein each bipolar pulse comprises a positive polarity voltage pulse and a negative polarity voltage pulse with a negligible delay between the positive polarity voltage pulse and the negative polarity voltage pulse, and (2) the third switch Q₃ to recover an energy stored in the inductor Lₘ. This design allows voltage to be boosted to a higher (or lower) value as desired. The negligible delay can be one microsecond or less, or no delay at all. Moreover, the one or more bipolar pulses can be a continueous stream of bipolar pulses with substantially no voltage degradation of the positive polarity voltage pulse and the negative polarity voltage pulse. As described below, the positive polarity voltage pulse and the negative polarity voltage pulse are preferably approximately rectangular in shape. Note that it is assumed that all circuit components are ideal for the purposes of this description.

The bipolar pulse generator 380 can use a single voltage source V₁. In addition, the controller 304 can be programmed using a graphical user interface to operate the first switch Q₁ and the second switch Q₂ selectively generate one or more unipolar pulses, or adjust the opening and closing of the first switch Q₁, the second switch Q₂ and the third switch Q₃ to change a duration of the positive polarity voltage pulse and the negative polarity voltage pulse. When the controller 304 opens the third switch Q₃ at an end of a source dominated discharge, approximately 50% of the energy stored in the inductor Lm is transferred to the cell or chamber 302 and approximately 50% of the energy stored in the inductor Lm is returned to the voltage source V₁. Note that the half-controlled bridge can be replaced with a full H-bridge by replacing the first diode D₁ with a fourth switch Q₄ and the second diode D₂ with a fifth switch Q₅ if the components can achieve the negligible delay. Although current switching technology may not be able to achieve the negibile delay using a full H-bridge design, future improvements to switching technology may make this a viable embodiment and is, therefore, within the scope of the present invention.

The cell or chamber 302 may contain an insulation, a biological sample, a medical sample, an environmental sample, an agricultural sample or a combination thereof. Likewise, the cell or chamber 302 may contains one or more biological cells such that bipolar pulses lyse the one or more biological cells such that one or more products, such as neutral lipids, proteins, triglycerides, sugars, and combinations and modifications thereof, are released. The neutral lipids, triglycerides or both can then be converted to yield a fatty acid methyl ester (FAME), a biodiesel or a biofuel. The one or more biological cells can be selected from a domain comprising Prokaryota and/or Eukaryota. The one or more biological cells can be selected from a division comprising Cyanophyta, Archaeplastida/Plantae sensu lato (includes the Phylum Viridiplantae, plants, which includes Chlorophyta, Rhodophyta, and Glaucophyta); Cabozoa (includes the Kingdom Excavata and Supergroup Rhizaria that represents the Euglenophyta and Chlorarachniophyta); Chromaveolata (includes the Supergroup Chromista and 20 Superphylum Aveolata that represent the Heterokontophyta, Haptophyta, Cryptophyta, and Dinophyta), as well as the Kingdom Fungi (all yeasts and fungal-related organisms). Moreover, the one or more biological cells can be algal cells, bacterial cells, viral cells or combinations thereof. Various algal cells are listed below.

The operation of the bipolar pulse generators 300, 340 and 380 will now be described.

Positive Pulse, 0≦t≦tₚ

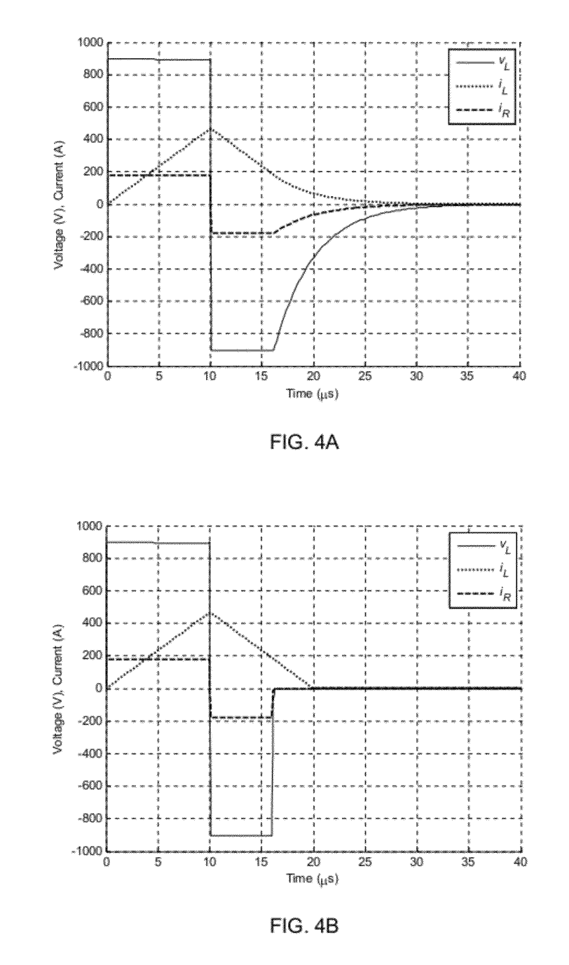

During bipolar operation Q₁ and Q₂ are always in the same state and are switched simultaneously; Q₃ is initially closed. Referring to FIG. 4A, when the switches are closed at time t=0 the source voltage V₁ is impressed across the load forming the positive pulse, and thus v_{L}=V₁, and i_{R}=V₁/R_{L}. The inductor Lₘ current i_{L} increases linearly to a maximum value, î_{L}, of

$${\hat{i}}_{L} = \frac{{V}_{1}}{{L}_{m}} {t}_{p} \qquad ( 1 )$$

where tₚ is the duration of the positive pulse (i.e. the time the switches are held closed).

Negative Pulse

When Q₁ and Q₂ are opened at time t=10 μs the load inductance begins discharging and thus v_{L}, and therefore i_{R} reverse polarity with no dead time before the reversal. Until t=16 μs, i_{L}, is greater than |i_{R}| and the negative pulse maintains a constant amplitude equal to that of the positive pulse. Excess inductor current discharges to the source through D₁ and D₂ providing for the recovery of some of the stored energy. This period of time is termed the source dominated discharge. After this time, a load dominated discharge occurs during which the load voltage decays to zero. Since this reducing voltage is ineffective in lysing algae, this discharge is considered an energy loss. To prevent this loss, Q₃ is opened at t=16 μs in FIG. 4B allowing the remaining stored inductor energy to return to the source thereby significantly enhancing the converter's efficiency.

Source Dominated Discharge, tₚ≦t≦tₚ+tₙ

As mentioned above, if while the switches were closed the inductor Lₘ was charged to a current value greater than the critical value of I_{L,critical}=V₁/R_{L} required by the load, excess energy is stored in the inductor and a source dominated discharge (SDD) will occur when the switches are opened. An SDD is one in which the discharge rate of Lₘ is controlled by the magnitude of the source voltage via the clamping action of D₁ and D₂. The chief benefit of an SDD is that the source voltage fixes the inductor discharge rate to a known and constant value independent of R_{L} yielding the desired pulse shape. Specifically,

$$\frac{d {i}_{L}}{d t} = - \frac{{V}_{1}}{{L}_{m}} . \qquad ( 2 )$$

While the discharge rate is independent of R_{L}, the negative pulse time, tₙ, is now as shown below:

$${t}_{n} = \frac{{\hat{i}}_{L} - {I}_{L , critical}}{d {i}_{L} / d t} = {t}_{p} - \frac{{L}_{m}}{{R}_{L}} . \qquad ( 3 )$$

Equation 3 leads to several important conclusions regarding the operation of the flyback converter during a source dominated discharge. First, because it must be true that tₙ≧0, values of Lₘ/R_{L}>tₚ will not result in a source dominated discharge, but rather will yield a load dominated discharge as will be discussed herein below. Second, tₙ can be no larger than tₚ; the positive and negative pulse durations are equal when R_{L} is an open circuit. Third, for a given value of R_{L}, tₙ approaches tₚ as Lₘ is decreased towards zero. In doing so the tradeoff is that grows proportionally with decreasing Lₘ according to Equation 1. The peak energy, Ŵ_{g}, stored in Lₘ however grows with the square of î_{L}, thus as Lₘ decreases, overall Ŵ_{g} increases proportionally with î_{L}. Therefore from the viewpoint of ensuring tₙ is as close to tₚ as possible, it is desirable to use the smallest value of Lₘ that maintains î_{L} below the maximum tolerable circuit component limit.

Load Dominated Discharge, t>tₚ+tₙ

When i_{L} falls below I_{L,critical} at the time t=tₚ+tₙ, the discharge of Lₘ is no longer dominated by the source but rather by the load. During a load dominated discharge (LDD) i_{L} is low enough such that when it flows through R_{L}, −v_{L} can no longer be raised above the source voltage. Therefore the diodes D₁ and D₂ become reverse biased and the source is unable to influence the discharge. Instead, the discharge of Lₘ is controlled solely by R_{L} with a time constant of

$$τ = \frac{{L}_{m}}{{R}_{L}} . \qquad ( 4 )$$

The voltage across the cell or chamber 302 is found as

v_{L}=−V₁e^{-tR}^{_{L}}^{/L}^{ₘ}. (5)

As mentioned hereinabove the decreasing magnitude of v_{L} during the LDD is ineffective in lysing algal cells and thus the energy, W_{LDD}, stored in the inductor at the beginning of the LDD (end of the SDD) is considered a loss. This loss can be avoided by opening Q₃ at the end of the SDD. Note for values of tₚ≦τ, tₙ=0 and there is no SDD.

Energy Usage During the Source Dominated Discharge

At the beginning of a source dominated discharge the energy stored in Lₘ is

$${\hat{W}}_{g} = \frac{1}{2} {L}_{m} {\hat{i}}_{mag}^{2} = \frac{1}{2} \frac{{V}_{1}^{2} {t}_{p}^{2}}{{L}_{m}} . \qquad ( 6 )$$

The energy W_{R} consumed by R_{L}, the energy W_{LDD} remaining in Lₘ at the end of the SDD, and the energy W_{rec} recovered by the source can be found respectively as:

$${W}_{R} = \frac{{V}_{1}^{2}}{{R}_{L}} ( {t}_{p} - \frac{{L}_{m}}{{R}_{L}} ) , {W}_{LDD} = \frac{1}{2} {{L}_{m} ( \frac{{V}_{1}}{{R}_{L}} )}^{2} , \\ {W}_{rec} = \frac{1}{2} \frac{{V}_{1}^{2}}{{L}_{m}} {( {t}_{p} - \frac{{L}_{m}}{{R}_{L}} )}^{2} . \qquad ( 7 )$$

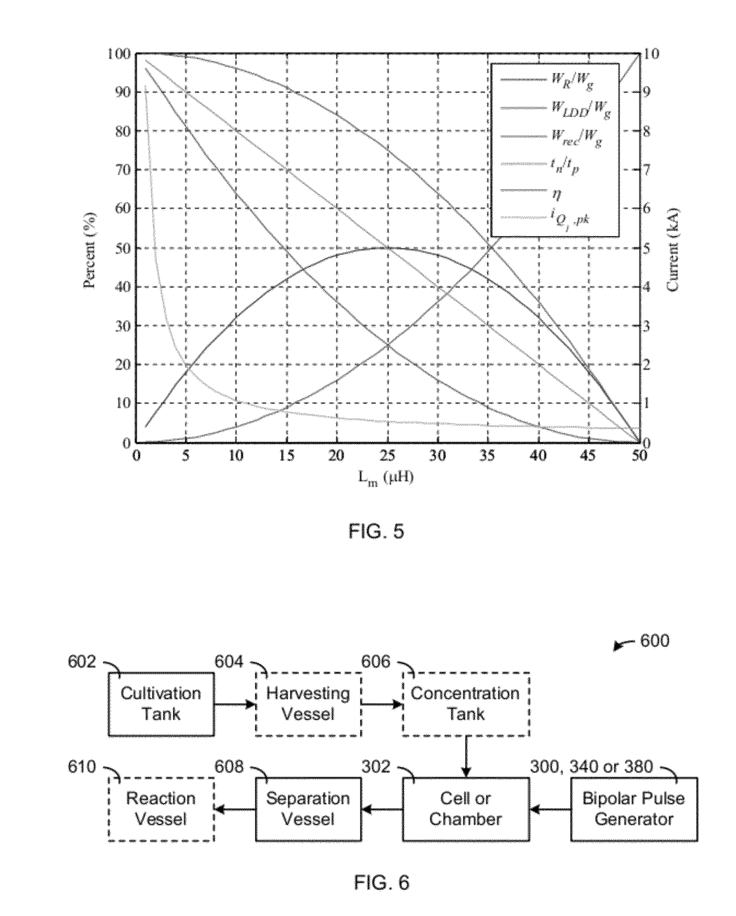

FIG. 5 shows W_{R}, W_{LDD}, W_{rec} at the end of a SDD, all normalized by Ŵ_{g} for various design inductor values. Also plotted are the negative pulse time normalized by the positive pulse time, the peak current in Q₁, and an efficiency estimate, η, determined as:

$$η = ( \frac{{\hat{W}}_{g} - {W}_{LDD}}{{\hat{W}}_{g}} ) \cdot 100 \% . \qquad ( 8 )$$

In Equation 8 it is assumed that energy recovered by the source incurs no losses (since the components are ideal) and that any energy that remains in the inductor after the SDD ends is a loss. Of primary importance in FIG. 5 is that the maximum portion of the energy stored in Lₘ that can be transferred to the load is 50%. At this operating point the source recovers 25% of the stored energy and 25% remains in the inductor after the SDD completes. Therefore, if Q₃ is not opened upon the completion of the SDD, the remaining 25% of the energy stored in Lₘ will be lost during the subsequent LDD. Under this condition the power supply efficiency is limited to 75% according to Equation 8. Somewhat improved efficiencies during the SDD can be obtained with only a moderate increase of the peak primary current and decrease of the energy percentage transferred to the load if Lₘ is designed to be slightly less than the value required for maximum energy transfer to the load. The greatest efficiency improvement is obtained by opening Q₃ at the end of the SDD. In doing so 50% of the inductor's stored energy is transferred to the load, and 50% is returned to the source. The theoretical maximum efficiency of the ideal flyback converter is the 100%. It is apparent that in a practical application the expected efficiency will necessarily be somewhat lower.

The flyback converter 300, 340 or 380 of the present invention are capable of unipolar operation in which only the positive voltage pulse is provided to the test cell. This modification is easily performed via a simple mode selection in the converter controller 304 whereby Q₂ is forced to remain closed at all times. Alternatively, a positive unipolar pulse can be created by leaving Q₃ open and operating Q₁ and Q₂ in synchronism as was done for the bipolar case. Compared to the method of producing positive pulses in which Q₂ is left open, this method is more efficient since energy stored in Lₘ at the end of the positive pulse is returned to the source via a SDD instead of being dissipated in the parasitic circuit resistances.

If desired the flyback converter 300, 340 or 380 can produce a bipolar pulse with a dead time between the pulses by opening only Q₁ after the inductor Lₘ is charged allowing the load current to circulate through D₂. During this time the dead time is created as the cell or chamber 302 voltage falls to very near zero volts. The negative pulse is produced in the usual manner when Q₂ is opened.

The three-switch bipolar flyback converter 300, 340 or 380 has been designed by the present inventors which is capable of producing bipolar rectangular voltage pulses across a cell or chamber 302 for the purpose of lysing algal cells contained within to extract the bio-oil. Compared to traditional bipolar pulsed power supplies, the rectangular output voltage of the flyback converter 300, 340 or 380 is able to swing directly from a positive polarity to a negative polarity with equal amplitudes, with no dead time in between, using only one voltage source, and without adjusting or reconnecting any of the converter's components. Moreover, the device is capable of outputting a continuous stream of pulses with no voltage amplitude degradation. Experimentation with algal cells suggests that the lysing of algal cells with such a waveform will be greatly enhanced allowing for significant savings in the production of algae-based bio-fuels. The absence of this delay improves the lysing of biological cells and therefore the oil yield. Increased oil yield decreases the fuel cost significantly, perhaps by 50% or more. Unlike other supplies it allows the user to select from a range of pulse patterns (bipolar with no delay, bipolar with delay, unipolar positive), and adjust the voltage amplitude and pulse duration to tailor the power supply to the biological cells to be lysed.

Furthermore, the three-switch bipolar flyback converter 300, 340 or 380 is highly efficient (thus allowing for economical fuel production from the lysis of biological cells like algal cells), in part, because it can recover unused energy within the power supply during operation. An active damping feature has also been included to ensure compatibility with a wide range of biological lysing test cell designs. This adjustability to various biological materials will allow more types of materials to be utilized for fuel making than present technology supplies allow. The design of the power supply is also easily scaled to meet the power requirements of the lysing desired. For instance, many current supplies cannot be used for medium or high power applications, and thus cannot perform large quantity lysing economically. The disclosed power supply is an inherently scalable architecture and thus can easily be tailored to essentially any biological lysing application. Due to the disclosed power supply's ability to transition between voltage polarities with no delay, it can be used wherever bipolar pulses are needed such as in insulation testing, other biological cell studies, and various medical, environmental, and agricultural applications.

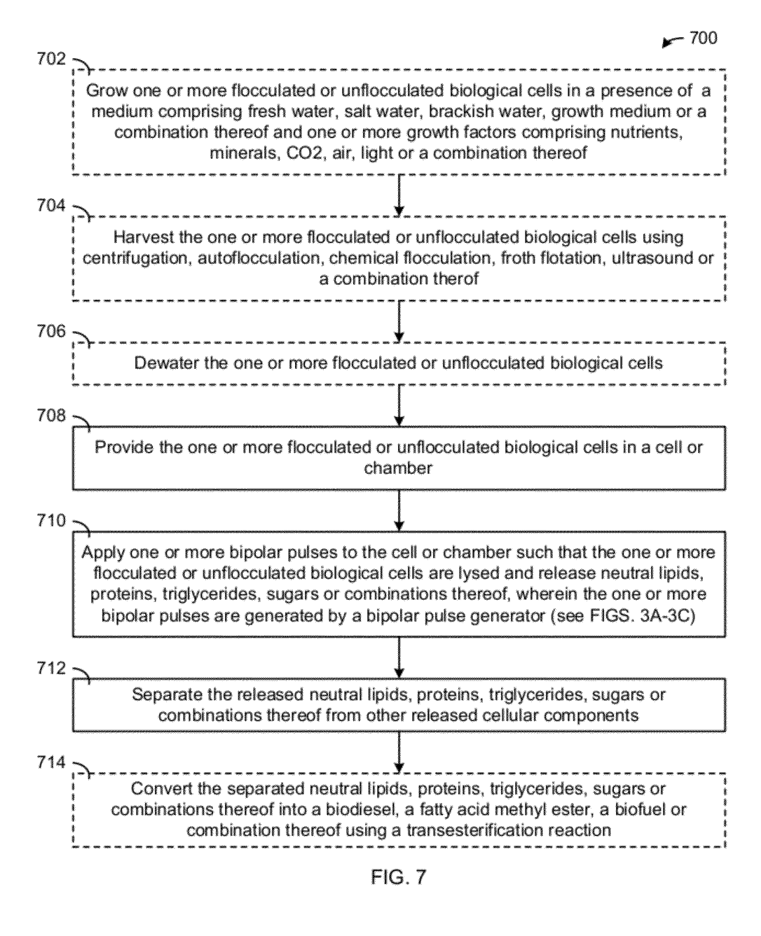

Now referring to FIG. 6, a block diagram of a system 600 for treating biological cells in accordance with another embodiment of the present invention is shown. The system 600 includes a cultivation tank 602, a cell or chamber 302 connected to the concentration tank 602, a bipolar pulse generator 300, 340 or 380 for delivering one or more bipolar pulses to the cell or chamber 302, and a separation vessel 608 connected to the cell or chamber 302. The cultivation tank 602 is used to grow the one or more flocculated or unflocculated biological cells in a presence of a medium comprising fresh water, salt water, brackish water, growth medium or a combination thereof and one or more growth factors comprising nutrients, minerals, CO₂, air, light or a combination thereof. The cell or chamber 302 is used for lysing the biological cells to release neutral lipids, proteins, triglycerides, sugars or combinations thereof using one or more bipolar pulses. The bipolar pulse generator 300 or 340 is described above in reference to FIGS. 3A-3C. The separation vessel 608 is used to separate the released neutral lipids, proteins, triglycerides, sugars or combinations thereof from other released cellular components.

Other components may include: a harvesting vessel 604 connected between the cultivation tank 602 and the cell or chamber 302 wherein the one or more flocculated or unflocculated biological cells are harvested using centrifugation, autoflocculation, chemical flocculation, froth flotation, ultrasound or a combination thereof; a concentration tank 606 connected between the cultivation tank 602 and the cell or chamber 302 wherein the one or more flocculated or unflocculated biological cells are dewatered; and/or a reaction vessel 610 connected to the separation vessel 608 for converting the separated neutral lipids, proteins, triglycerides, sugars or combinations thereof into a biodiesel, a fatty acid methyl ester, a biofuel or combination thereof using a transesterification reaction.

Referring now to FIG. 7, a flow chart of a method 700 for treating biological cells in accordance with another embodiment of the present invention is shown. The one or more flocculated or unflocculated biological cells in a cell or chamber 302 are provided in block 708. One or more bipolar pulses are applied to the cell or chamber 302 in block 710 such that the one or more flocculated or unflocculated biological cells are lysed and release neutral lipids, proteins, triglycerides, sugars or combinations thereof, wherein the one or more bipolar pulses are generated by the bipolar pulse generator 300, 340 or 380 as described above in reference to FIGS. 3A-3C. The released neutral lipids, proteins, triglycerides, sugars or combinations thereof are separated from other released cellular components in block 712.

Other steps may include: growing the one or more flocculated or unflocculated biological cells in a presence of a medium comprising fresh water, salt water, brackish water, growth medium or a combination thereof and one or more growth factors comprising nutrients, minerals, CO₂, air, light or a combination thereof in block 702; harvesting the one or more flocculated or unflocculated biological cells using centrifugation, autoflocculation, chemical flocculation, froth flotation, ultrasound or a combination thereof in block 704; dewatering the one or more flocculated or unflocculated biological cells in block 706; and/or converting the separated neutral lipids, proteins, triglycerides, sugars or combinations thereof into a biodiesel, a fatty acid methyl ester, a biofuel or combination thereof using a transesterification reaction in block 714.

To facilitate the understanding of this invention, a number of terms are defined below. Terms defined herein have meanings as commonly understood by a person of ordinary skill in the areas relevant to the present invention. The terminology herein is used to describe specific embodiments of the invention, but their usage does not limit the invention, except as outlined in the claims.

As used herein the term “algae” represents a large, heterogeneous group of primitive photosynthetic organisms, which occur throughout all types of aquatic habitats and moist terrestrial environments. Nadakavukaren et al., Botany. An Introduction to Plant Biology, 324-325, (1985). The term “algae” as described herein is intended to include the species selected from the group consisting of the diatoms (bacillariophytes), green algae (chlorophytes), blue-green algae (cyanophytes), golden-brown algae (chrysophytes), haptophytes, freshwater algae, saltwater algae, Amphipleura, Amphora, Chaetoceros, Cyclotella, Cymbella, Fragilaria, Hantzschia, Navicula, Nitzschia, Phaeodactylum, Thalassiosira Ankistrodesmus, Botryococcus, Chlorella, Chlorococcum, Dunaliella, Monoraphidium, Oocystis, Scenedesmus, Nanochloropsis, Tetraselmis, Chlorella, Dunaliella, Oscillatoria, Synechococcus, Boekelovia, Isochysis and Pleurochysis.

Some non-limiting examples of the divisons of algae that may be used in the present invention include Chlorophyta, Cyanophyta (Cyanobacteria), Rhodophyta (red algae), and Heterokontophyt. Non-limiting examples of classes of microalgae that may be used with the present invention include: Bacillariophyceae, Eustigmatophyceae, and Chrysophyceae. Non-limiting examples of genera of microalgae used with the methods of the invention include: Nannochloropsis, Chlorella, Dunaliella, Scenedesmus, Selenastrum, Oscillatoria, Phormidium, Spirulina, Amphora, and Ochromonas. Non-limiting examples of microalgae species that can be used with the present invention include: Achnanthes orientalis, Agmenellum spp., Amphiprora hyaline, Amphoracoffeiformis, Amphora coffeiformis var. linea, Amphora coffeiformis var. punctata, Amphora coffeiformis var. taylori, Amphora coffeiformis var. tenuis, Amphora delicatissima, Amphora delicatissima var. capitata, Amphora sp., Anabaena, Ankistrodesmus, Ankistrodesmus falcatus, Boekelovia hooglandii, Borodinella sp., Botryococcus braunii, Botryococcus sudeticus, Bracteococcus minor, Bracteococcus medionucleatus, Carteria, Chaetoceros gracilis, Chaetoceros muelleri, Chaetoceros muelleri var. subsalsum, Chaetoceros sp., Chlamydomas perigranulata, Chlorella anitrata, Chlorella antarctica, Chlorella aureoviridis, Chlorella candida, Chlorella capsulate, Chlorella desiccate, Chlorella ellipsoidea, Chlorella emersonii, Chlorella fusca, Chlorella fusca var. vacuolata, Chlorella glucotropha, Chlorella infusionum, Chlorella infusionum var. actophila, Chlorella infusionum var. auxenophila, Chlorellakessleri, Chlorella lobophora, Chlorella luteoviridis, Chlorella luteoviridis var. aureoviridis, Chlorella luteoviridis var. lutescens, Chlorella miniata, Chlorella minutissima, Chlorella mutabilis, Chlorella nocturna, Chlorella ovalis, Chlorella parva, Chlorella photophila, Chlorella pringsheimii, Chlorella protothecoides, Chlorella protothecoides var. acidicola, Chlorella regularis, Chlorella regularis var. minima, Chlorella regularis var. umbricata, Chlorella reisiglii, Chlorella saccharophila, Chlorella saccharophila var. ellipsoidea, Chlorella salina, Chlorella simplex, Chlorella sorokiniana, Chlorella sp., Chlorella sphaerica, Chlorella stigmatophora, Chlorella vanniellii, Chlorella vulgaris, Chlorella vulgaris fo. tertia, Chlorella vulgaris var. autotrophica, Chlorella vulgaris var. viridis, Chlorella vulgaris var. vulgaris, Chlorella vulgaris var. vulgaris fo. tertia, Chlorella vulgaris var. vulgaris fo. viridis, Chlorella xanthella, Chlorella zofingiensis, Chlorella trebouxioides, Chlorella vulgaris, Chlorococcum infusionum, Chlorococcum sp., Chlorogonium, Chroomonas sp., Chrysosphaera sp., Cricosphaera sp., Crypthecodinium cohnii, Cryptomonas sp., Cyclotella cryptica, Cyclotella meneghiniana, Cyclotella sp., Dunaliella sp., Dunaliella bardawil, Dunaliella bioculata, Dunaliella granulate, Dunaliella maritime, Dunaliella minuta, Dunaliella parva, Dunaliella peircei, Dunaliella primolecta, Dunaliella salina, Dunaliella terricola, Dunaliella tertiolecta, Dunaliella viridis, Dunaliella tertiolecta, Eremosphaera viridis, Eremosphaera sp., Effipsoidon sp., Euglena spp., Franceia sp., Fragilaria crotonensis, Fragilaria sp., Gleocapsa sp., Gloeothamnion sp., Haematococcus pluvialis, Hymenomonas sp., lsochrysis aff. galbana, lsochrysis galbana, Lepocinclis, Micractinium, Micractinium, Monoraphidium minutum, Monoraphidium sp., Nannochloris sp., Nannochloropsissalina, Nannochloropsis sp., Navicula acceptata, Navicula biskanterae, Navicula pseudotenelloides, Navicula pelliculosa, Navicula saprophila, Navicula sp., Nephrochloris sp., Nephroselmis sp., Nitschia communis, Nitzschia alexandrina, Nitzschia closterium, Nitzschia communis, Nitzschia dissipata, Nitzschia frustulum, Nitzschia hantzschiana, Nitzschia inconspicua, Nitzschia intermedia, Nitzschia microcephala, Nitzschia pusilla, Nitzschia pusilla elliptica, Nitzschia pusilla monoensis, Nitzschia quadrangular, Nitzschia sp., Ochromonas sp., Oocystis parva, Oocystis pusilla, Oocystis sp., Oscillatoria limnetica, Oscillatoria sp., Oscillatoria subbrevis, Parachlorella kessleri, Pascheriaacidophila, Pavlova sp., Phaeodactylum tricomutum, Phagus, Phormidium, Platymonas sp., Pleurochrysis carterae, Pleurochrysis dentate, Pleurochrysis sp., Prototheca wickerhamii, Prototheca stagnora, Prototheca portoricensis, Prototheca moriformis, Prototheca zopfii, Pseudochlorella aquatica, Pyramimonas sp., Pyrobotrys, Rhodococcus opacus, Sarcinoid chrysophyte, Scenedesmus armatus, Schizochytrium, Spirogyra, Spirulina platensis, Stichococcus sp., Synechococcus sp., Synechocystisf, Tagetes erecta, Tagetes patula, Tetraedron, Tetraselmis sp., Tetraselmis suecica, Thalassiosira weissflogii, and Viridiella fridericiana.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C or combinations thereof” is intended to include at least one of: A, B, C, AB, AC, BC or ABC, and if order is important in a particular context, also BA, CA, CB, CBA, BCA, ACB, BAC or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

It will be understood that particular embodiments described herein are shown by way of illustration and not as limitations of the invention. All of the devices, systems and/or methods disclosed and claimed herein can be made and executed without undue experimentation in light of the present disclosure. Likewise, the various illustrative logical blocks, modules, circuits, and algorithm steps described herein may be implemented as electronic hardware, computer software, or combinations of both, depending on the application and functionality. Moreover, the various logical blocks, modules, and circuits described herein may be implemented or performed with a general purpose processor (e.g., microprocessor, conventional processor, controller, microcontroller, state machine or combination of computing devices), a digital signal processor (“DSP”), an application specific integrated circuit (“ASIC”), a field programmable gate array (“FPGA”) or other programmable logic device, discrete gate or transistor logic, discrete hardware components, or any combination thereof designed to perform the functions described herein. Similarly, steps of a method or process described herein may be embodied directly in hardware, in a software module executed by a processor, or in a combination of the two. A software module may reside in RAM memory, flash memory, ROM memory, EPROM memory, EEPROM memory, registers, hard disk, a removable disk, a CD-ROM, or any other form of storage medium known in the art.

The principal features of this invention can be employed in various embodiments without departing from the spirit, scope and concept of the invention. Those skilled in the art will recognize or be able to ascertain using no more than routine experimentation, numerous equivalents to the specific procedures described herein. All such substitutions, modifications and equivalents to those skilled in the art are deemed to be within the spirit, scope and concept of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

### REFERENCES

U.S. Patent Application Publication No. 2009/0087900: Apparatus for Performing Electrodistention on Algae Cells.
U.S. Pat. No. 6,043,066: Cell Separation using Electric Fields.
U.S. Patent Application Publication No. 2012/0021481: Electromechanical Lysing Of Algae Cells.
Kolb, J. F. Kono, S. Schoenbach, K. H., “Nanosecond pulsed electric field generators for the study of subcellular effects”, BIOELECTROMAGNETICS, 2006, Vol. 27, No. 3, pages 172-187.
Redondo, L. M. Canacsinh, H. Silva, J. F., “Generalized Solid-state Marx Modulator Topology”, IEEE Transactions on Dielectrics and Electrical Insulation”, August 2009, Vol. 16, No. 4.
K. Jong-Hyun, L. Sang-Cheol, L. Byoung-Kuk, S. V. Shenderey, K. Jong-Soo, and R. Geun-Hie, “A high-voltage bi-polar pulse generator a using push-pull inverter”, Proc. Industrial Electronics Society Conference, 2003, vol. 1, pp. 102-107.
M. Petkovsek, P. Zajec, J. Nastran, and D. Voncina, “Multilevel bipolar high voltage pulse source—interlock dead time reduction”, EUROCON 2003, vol. 2, pp. 240-243.
Behrend, M. Kuthi, A. Gu, X. Vernier, P. T. Marcu, L. Craft, C. M. Gundersen, M. A., “Pulse Generators for Pulsed Electric Field Exposure of Biological Cells and Tissues”, IEEE Transactions on Dielectrics and Electrical Insulation, 2003, Vol. 10; PART 5, pages 820-825.
Sanders, 1. M. Kuthi, A. Wu, Y., Vernier, T. Gundersen, M. A., “A Linear, Single-stage, Nanosecond Pulse Generator for Delivering Intense Electric Fileds to Bilogical Loads”, IEEE Transactions on Dielectrics and Electrical Insulation, August 2009, Vol. 16, No. 3.
Grenier, 1. R. Jayaram, S. H. Kazerani, M. Wang, H. Griffiths, M. W., “MOSFET-Based Pulse Power Supply for Bacterial Transformation”, IEEE Transactions on Inducstry Applications, 2008, Vol. 44; No. 1, pages 25-31.
Ghasemi, Z. Macgregor, S. Anderson, J. Lamont, Y., “Development of an integrated solid-state generator for light inactivation of food-related pathogenic bacteria”, Institute of Physics Measurement Science and Technology, 2003, Vol. 14, PART 6, pages N26-N32.

What is claimed is:

1. A bipolar pulse generator comprising:
a voltage source;
a half-controlled bridge connected to the voltage source, wherein the half-controlled bridge comprises a first switch, a second switch, a first diode and a second diode;
a load connected across the half-controlled bridge, wherein the load comprises an inductor connected in parallel with a cell or chamber; and
a controller connected to the first switch and second switch, wherein the controller operates the first and second switches to selectively generate one or more bipolar pulses, wherein each bipolar pulse comprises a positive polarity voltage pulse and a negative polarity voltage pulse with a negligible delay between the positive polarity voltage pulse and the negative polarity voltage pulse.

2. The bipolar pulse generator of claim 1, further comprising an energy recovery circuit connected in series with the cell or chamber such that the cell or chamber and the energy recovery circuit are connected in parallel with the inductor.

3. The bipolar pulse generator of claim 2, wherein the energy recovery circuit comprises a third diode connected in parallel with a third switch, and the controller operates the third switch to recover an energy stored in the inductor.

4. The bipolar pulse generator of claim 3, wherein the third switch is opened at an end of a source dominated discharge such that approximately 50% of the energy stored in the inductor is transferred to the cell or chamber and approximately 50% of the energy stored in the inductor is returned to the voltage source.

5. The bipolar pulse generator of claim 1, wherein the inductor comprises a transformer, wherein a primary winding of the transformer is connected across the half-controlled bridge and a secondary winding is connected in parallel with the cell or chamber.

6. The bipolar pulse generator of claim 5, wherein the transformer has one or more taps for changing a voltage across the secondary winding.

7. The bipolar pulse generator of claim 1, wherein the half-controlled bridge is replaced with a full H-bridge such that the first diode is replaced with a fourth switch and the second diode is replaced with a fifth switch.

8. The bipolar pulse generator of claim 1, wherein the negligible delay comprises a delay of one microsecond or less.

9. The bipolar pulse generator of claim 1, wherein the negligible delay comprises no delay.

10. The bipolar pulse generator of claim 1, wherein the one or more bipolar pulses comprise a continueous stream of bipolar pulses with substantially no voltage degradation of the positive polarity voltage pulse and the negative polarity voltage pulse.

11. The bipolar pulse generator of claim 1, wherein the positive polarity voltage pulse and the negative polarity voltage pulse are approximately rectangular.

12. The bipolar pulse generator of claim 1, wherein the voltage source comprises a single voltage source.

13. The bipolar pulse generator of claim 1, wherein the first switch and the second switch comprise transistors or thyristors.

14. The bipolar pulse generator of claim 1, wherein the controller further operates the first and second switches to selectively generate one or more unipolar pulses.

15. The bipolar pulse generator of claim 1, wherein the controller adjusts the opening and closing of the first switch and the second switch to change a duration of the positive polarity voltage pulse and the negative polarity voltage pulse.

16. The bipolar pulse generator of claim 1, wherein the cell or chamber contains an insulation, a biological sample, a medical sample, an environmental sample, an agricultural sample or a combination thereof.

17. The bipolar pulse generator of claim 1, wherein the cell or chamber contains one or more biological cells and the bipolar pulses lyse the one or more biological cells.

18. The bipolar pulse generator of claim 17, wherein the one or more biological cells on lysis release one or more products selected from the group consisting of neutral lipids, proteins, triglycerides, sugars, and combinations and modifications thereof.

19. The bipolar pulse generator of claim 18, wherein the neutral lipids, triglycerides or both are converted to yield a fatty acid methyl ester (FAME), a biodiesel or a biofuel.

20. The bipolar pulse generator of claim 17, wherein the one or more biological cells comprise algal cells, bacterial cells, viral cells or combinations thereof.

21. The bipolar pulse generator of claim 17, wherein the one or more biological cells are selected from a domain comprising Prokaryota and/or Eukaryota.

22. The bipolar pulse generator of claim 17, wherein one or more biological cells are selected from a division comprising Cyanophyta, Archaeplastida/Plantae sensu lato (includes the Phylum Viridiplantae, plants, which includes Chlorophyta, Rhodophyta, and Glaucophyta); Cabozoa (includes the Kingdom Excavata and Supergroup Rhizaria that represents the Euglenophyta and Chlorarachniophyta); Chromaveolata (includes the Supergroup Chromista and 20 Superphylum Aveolata that represent the Heterokontophyta, Haptophyta, Cryptophyta, and Dinophyta), as well as the Kingdom Fungi (all yeasts and fungal-related organisms).

23. A system for treating one or more flocculated or unflocculated biological cells comprising:
a cultivation tank for growing the one or more flocculated or unflocculated biological cells in a presence of a medium comprising fresh water, salt water, brackish water, growth medium or a combination thereof and one or more growth factors comprising nutrients, minerals, CO₂, air, light or a combination thereof;
a cell or chamber connected to the cultivation tank for lysing the one or more flocculated or unflocculated biological cells to release neutral lipids, proteins, triglycerides, sugars or combinations thereof using one or more bipolar pulses;
a bipolar pulse generator comprising:
a voltage source,
a half-controlled bridge connected to the voltage source, wherein the half-controlled bridge comprises a first switch, a second switch, a first diode and a second diode,
a load connected across the half-controlled bridge, wherein the load comprises an inductor connected in parallel with the cell or chamber, and
a controller connected to the first switch and the second switch, wherein the controller operates the first and second switches to selectively generate the one or more bipolar pulses, wherein each bipolar pulse comprises a positive polarity voltage pulse and a negative polarity voltage pulse with a negligible delay between the positive polarity voltage pulse and the negative polarity voltage pulse; and
a separation vessel connected to the cell or chamber for separating the released neutral lipids, proteins, triglycerides, sugars or combinations thereof from other released cellular components.

24. The system of claim 23, further comprising a harvesting vessel connected between the cultivation tank and the cell or chamber wherein the one or more flocculated or unflocculated biological cells are harvested using centrifugation, autoflocculation, chemical flocculation, froth flotation, ultrasound or a combination thereof.

25. The system of claim 23, further comprising a concentration tank connected between the cultivation tank and the cell or chamber wherein the one or more flocculated or unflocculated biological cells are dewatered.

26. The system of claim 23, further comprising a reaction vessel connected to the separation vessel for converting the separated neutral lipids, proteins, triglycerides, sugars or combinations thereof into a biodiesel, a fatty acid methyl ester, a biofuel or combination thereof using a transesterification reaction.

27. The system of claim 23, further comprising an energy recovery circuit connected in series with the cell or chamber such that the cell or chamber and the energy recovery circuit are connected in parallel with the inductor.

28. The system of claim 27, wherein the energy recovery circuit comprises a third diode connected in parallel with a third switch, and the controller operates the third switch to recover an energy stored in the inductor.

29. The system of claim 28, wherein the third switch is opened at an end of a source dominated discharge such that approximately 50% of the energy stored in the inductor is transferred to the cell or chamber and approximately 50% of the energy stored in the inductor is returned to the voltage source.

30. The system of claim 23, wherein the inductor comprises a transformer, wherein a primary winding of the transformer is connected across the half-controlled bridge and a secondary winding is connected in parallel with the cell or chamber.

31. The system of claim 30, wherein the transformer has one or more taps for changing a voltage across the secondary winding.

32. The system of claim 23, wherein the half-controlled bridge is replaced with a full H-bridge such that the first diode is replaced with a fourth switch and the second diode is replaced with a fifth switch.

33. The system of claim 23, wherein the negligible delay comprises a delay of one microsecond or less.

34. The system of claim 23, wherein the negligible delay comprises no delay.

35. The system of claim 23, wherein the one or more bipolar pulses comprise a continueous stream of bipolar pulses with substantially no voltage degradation of the positive polarity voltage pulse and the negative polarity voltage pulse.

36. The system of claim 23, wherein the positive polarity voltage pulse and the negative polarity voltage pulse are approximately rectangular.

37. The system of claim 23, wherein the voltage source comprises a single voltage source.

38. The system of claim 23, wherein the first switch and the second switch comprise transistors or thyristors.

39. The system of claim 23, wherein the controller further operates the first and second switches to selectively generate one or more unipolar pulses.

40. The system of claim 23, wherein the controller adjusts the opening and closing of the first switch and the second switch to change a duration of the positive polarity voltage pulse and the negative polarity voltage pulse.

41. The system of claim 23, wherein the one or more biological cells comprise algal cells, bacterial cells, viral cells or combinations thereof.

42. The system of claim 23, wherein the one or more biological cells are selected from a domain comprising Prokaryota and/or Eukaryota.

43. The system of claim 23, wherein one or more biological cells are selected from a division comprising Cyanophyta, Archaeplastida/Plantae sensu lato (includes the Phylum Viridiplantae, plants, which includes Chlorophyta, Rhodophyta, and Glaucophyta); Cabozoa (includes the Kingdom Excavata and Supergroup Rhizaria that represents the Euglenophyta and Chlorarachniophyta); Chromaveolata (includes the Supergroup Chromista and 20 Superphylum Aveolata that represent the Heterokontophyta, Haptophyta, Cryptophyta, and Dinophyta), as well as the Kingdom Fungi (all yeasts and fungal-related organisms).

44. A method for treating one or more flocculated or unflocculated biological cells comprising the steps of:
providing the one or more flocculated or unflocculated biological cells in a cell or chamber;
applying one or more bipolar pulses to the cell or chamber such that the one or more flocculated or unflocculated biological cells are lysed and release neutral lipids, proteins, triglycerides, sugars or combinations thereof, wherein the one or more bipolar pulses are generated by:
a voltage source,
a half-controlled bridge connected to the voltage source, wherein the half-controlled bridge comprises a first switch, a second switch, a first diode and a second diode,
a load connected across the half-controlled bridge, wherein the load comprises an inductor connected in parallel with the cell or chamber connected, and
a controller connected to the first switch, second switch and the third switch, wherein the controller operates the first and second switches to selectively generate the one or more bipolar pulses, wherein each bipolar pulse comprises a positive polarity voltage pulse and a negative polarity voltage pulse with a negligible delay between the positive polarity voltage pulse and the negative polarity voltage pulse; and
separating the released neutral lipids, proteins, triglycerides, sugars or combinations thereof from other released cellular components.

45. The method of claim 44, further comprising the step of growing the one or more flocculated or unflocculated biological cells in a presence of a medium comprising fresh water, salt water, brackish water, growth medium or a combination thereof and one or more growth factors comprising nutrients, minerals, CO₂, air, light or a combination thereof.

46. The method of claim 44, further comprising the step of harvesting the one or more flocculated or unflocculated biological cells using centrifugation, autoflocculation, chemical flocculation, froth flotation, ultrasound or a combination thereof.

47. The method of claim 44, further comprising the step of dewatering the one or more flocculated or unflocculated biological cells.

48. The method of claim 44, further comprising the step of converting the separated neutral lipids, proteins, triglycerides, sugars or combinations thereof into a biodiesel, a fatty acid methyl ester, a biofuel or combination thereof using a transesterification reaction.

49. The method of claim 44, further comprising an energy recovery circuit connected in series with the cell or chamber such that the cell or chamber and the energy recovery circuit are connected in parallel with the inductor.

50. The method of claim 49, wherein the energy recovery circuit comprises a third diode connected in parallel with a third switch, and the controller operates the third switch to recover an energy stored in the inductor.

51. The method of claim 50, wherein the third switch is opened at an end of a source dominated discharge such that approximately 50% of the energy stored in the inductor is transferred to the cell or chamber and approximately 50% of the energy stored in the inductor is returned to the voltage source.

52. The method of claim 44, wherein the inductor comprises a transformer, wherein a primary winding of the transformer is connected across the half-controlled bridge and a secondary winding is connected in parallel with the cell or chamber.

53. The method of claim 53, wherein the transformer has one or more taps for changing a voltage across the secondary winding.

54. The method of claim 44, wherein the half-controlled bridge is replaced with a full H-bridge such that the first diode is replaced with a fourth switch and the second diode is replaced with a fifth switch.

55. The method of claim 44, wherein the negligible delay comprises a delay of one microsecond or less.

56. The method of claim 44, wherein the negligible delay comprises no delay.

57. The method of claim 44, wherein the one or more bipolar pulses comprise a continueous stream of bipolar pulses with substantially no voltage degradation of the positive polarity voltage pulse and the negative polarity voltage pulse.

58. The method of claim 44, wherein the positive polarity voltage pulse and the negative polarity voltage pulse are approximately rectangular.

59. The method of claim 44, wherein the voltage source comprises a single voltage source.

60. The method of claim 44, wherein the first switch and the second switch comprise transistors or thyristors.

61. The method of claim 44, wherein the controller further operates the first and second switches to selectively generate one or more unipolar pulses.

62. The method of claim 44, wherein the controller adjusts the opening and closing of the first switch and the second switch to change a duration of the positive polarity voltage pulse and the negative polarity voltage pulse.

63. The method of claim 44, wherein the one or more bipolar pulses are generated by the steps of:
applying a voltage from the voltage source while keeping the first switch and the second switch closed to produce the positive pulse across the load and charge the inductor; and
reversing a polarity of the positive pulse to form a negative pulse of a constant amplitude and equal to the positive pulse by opening the first switch and the second switch resulting in a current discharge from the inductor through the first diode and the second diode.

64. The method of claim 44, wherein the one or more biological cells comprise algal cells, bacterial cells, viral cells or combinations thereof.

65. The method of claim 44, wherein the one or more biological cells are selected from a domain comprising Prokaryota and/or Eukaryota.

66. The method of claim 44, wherein one or more biological cells are selected from a division comprising Cyanophyta, Archaeplastida/Plantae sensu lato (includes the Phylum Viridiplantae, plants, which includes Chlorophyta, Rhodophyta, and Glaucophyta); Cabozoa (includes the Kingdom Excavata and Supergroup Rhizaria that represents the Euglenophyta and Chlorarachniophyta); Chromaveolata (includes the Supergroup Chromista and 20 Superphylum Aveolata that represent the Heterokontophyta, Haptophyta, Cryptophyta, and Dinophyta), as well as the Kingdom Fungi (all yeasts and fungal-related organisms).
